SEQUENCE LISTING (6 sequences)

SEQ ID NO: 1 - DNA, 1685 bp, Danio rerio
  CDS (1)...(1389)
atggcagcgcaggtggcaaccacaaaggttccggaggtgcgggacatcactcgcattgagagaatcggcgctcactctcacattcgtggccttgggttggatgatgctctggagccacgacaggtgtctcaggggatggtgggccagctggcatctcgaagggcagccgggctgattcttgaaatgatcaaagatggtcagattgctggtcgcgcagtccttattgctggccaacctgggacgggcaagacagctattgctatgggtattgcccagtcgcttggtcctgacacacctttcactgctctggctgggagtgagattttctctctggagatgagcaagactgaggctttgagccaagcattccgcaaggccataggagtcagaataaaagaggagacggagatcattgaaggagaggtggtagagattcagattgacagacctgctacaggaacaggtgcaaaagttggtaaactcactctgaagacgacagagatggaaaccatatatgatctggggaccaagatgatcgagagcctcagtaaagagagagtacaagccggagatgtgattactattgataaagccacagggaagattagcaagcttggacggtcatttactcgagcaagagactatgatgccatgggagcacagacgcagtttgtgcagtgtccagagggggaacttcaaaagaggaaggaggttgttcacaccgtctctcttcatgaaattgacgtcatcaacagccgcacacagggattcttggccttattttcaggcgacacaggagagattaagtcagaggttcgggagcagatcaatgctaaagtgtctgaatggagagaggaaggcaaagctgaaattattcctggggttctcttcatagacgaggtccacatgctggacatagaatgcttttcattcctgaaccgagccctagagagtgatctttctcctgtactcatcatggctaccaacagaggcatcactcgaattagaggcaccaattaccagagtcctcatggtattcccatagacatgctggaccgtctgcttatcatcgctactactccttacacagagaaagagaccagacagatcctaaagattcgctgtgaggaggaggatgttgagctcagtgaagaggcacatacagttttaacacgtatcggacaggagacttcactccgctacgccatccagctcatcagtactgcaggcctggtgtgccgtaaacgcaggggcacagaagtccaagtggaggacattaaaagggtttattctctcttcctggatgaggccaggtcctcacagtacatgaaagagtatcaggactccttcctcttcaatgaaacacaaacctctcagatggacacctcgtaacgagtggagctgtgaataagcatgtccagtcattgctcctgtgaagacctttctcttatgacctctcctagtgctgagccatctctcaagtgttcaggcaattcagtgttcatgcactcaaatcctcatcttcaagtcctttttttttttttttttttgctattgctagattttatatgttttgcaatgaataaaaataatttcttcttgtttggctgtataacatggcaacttaaaggatgaaataaaagatatttgcccataaaaaaaaaaaaaaaaaaaaaaaaaaaaaa

SEQ ID NO: 2 - protein, 463 aa, Danio rerio
MAAQVATTKVPEVRDITRIERIGAHSHIRGLGLDDALEPRQVSQGMVGQLASRRAAGLILEMIKDGQIAGRAVLIAGQPGTGKTAIAMGIAQSLGPDTPFTALAGSEIFSLEMSKTEALSQAFRKAIGVRIKEETEIIEGEVVEIQIDRPATGTGAKVGKLTLKTTEMETIYDLGTKMIESLSKERVQAGDVITIDKATGKISKLGRSFTRARDYDAMGAQTQFVQCPEGELQKRKEVVHTVSLHEIDVINSRTQGFLALFSGDTGEIKSEVREQINAKVSEWREEGKAEIIPGVLFIDEVHMLDIECFSFLNRALESDLSPVLIMATNRGITRIRGTNYQSPHGIPIDMLDRLLIIATTPYTEKETRQILKIRCEEEDVELSEEAHTVLTRIGQETSLRYAIQLISTAGLVCRKRRGTEVQVEDIKRVYSLFLDEARSSQYMKEYQDSFLFNETQTSQMDTS

SEQ ID NO: 3 - DNA, 1488 bp, Danio rerio
  CDS (14)...(1402)
gttggtgagcatcatggcaaccgttacagccacaaccaaagtcccggagatccgtgatgtaacaaggattgagcgaatcggtgcccactcccacatccggggactggggctggacgatgccttggagcctcggcaggcttcgcaaggcatggtgggtcagctggcggcacggcgggcggctggcgtggtgctggagatgatccgggaagggaagattgccggtcgggcagtccttattgctggccagccgggcacggggaagacggccatcgccatgggcatggcgcaggccctgggccctgacacgccattcacagccatcgccggcagtgaaatcttctccctggagatgagcaagaccgaggcgctgacgcaggccttccggcggtccatcggcgttcgcatcaaggaggagacggagatcatcgaaggggaggtggtggagatccagattgatcgaccagcaacagggacgggctccaaggtgggcaaactgaccctcaagaccacagagatggagaccatctacgacctgggcaccaagatgattgagtccctgaccaaggacaaggtccaggccggggacgtgatcaccatcgacaaggcgacgggcaagatctccaagctgggccgctccttcacacgcgcccgcgactacgacgctatgggctcccagaccaagttcgtgcagtgcccagatggggagctccagaaacgcaaggaggtggtgcacaccgtgtccctgcacgagatcgacgtcatcaactctcgcacccagggcttcctggcgctcttctcaggtgacacaggggagatcaagtcagaagtccgtgagcagatcaatgccaaggtggctgagtggcgcgaggagggcaaggcggagatcatccctggagtgctgttcatcgacgaggtccacatgctggacatcgagagcttctccttcctcaaccgggccctggagagtgacatggcgcctgtcctgatcatggccaccaaccgtggcatcacgcgaatccggggcaccagctaccagagccctcacggcatccccatagacctgctggaccggctgcttatcgtctccaccaccccctacagcgagaaagacacgaagcagatcctccgcatccggtgcgaggaagaagatgtggagatgagtgaggacgcctacacggtgctgacccgcatcgggctggagacgtcactgcgctacgccatccagctcatcacagctgccagcttggtgtgccggaaacgcaagggtacagaagtgcaggtggatgacatcaagcgggtctactcactcttcctggacgagtcccgctccacgcagtacatgaaggagtaccaggacgccttcctcttcaacgaactcaaaggcgagaccatggacacctcctgagttggatgtcatcccccgaccccaccctgttttccaccagagttctgacactgtgactctgtataaaatggttgggaagctgc

SEQ ID NO: 4 - protein, 463 aa, Danio rerio
MATVTATTKVPEIRDVTRIERIGAHSHIRGLGLDDALEPRQASQGMVGQLAARRAAGVVLEMIREGKIAGRAVLIAGQPGTGKTAIAMGMAQALGPDTPFTAIAGSEIFSLEMSKTEALTQAFRRSIGVRIKEETEIIEGEVVEIQIDRPATGTGSKVGKLTLKTTEMETIYDLGTKMIESLTKDKVQAGDVITIDKATGKISKLGRSFTRARDYDAMGSQTKFVQCPDGELQKRKEVVHTVSLHEIDVINSRTQGFLALFSGDTGEIKSEVREQINAKVAEWREEGKAEIIPGVLFIDEVHMLDIESFSFLNRALESDMAPVLIMATNRGITRIRGTSYQSPHGIPIDLLDRLLIVSTTPYSEKDTKQILRIRCEEEDVEMSEDAYTVLTRIGLETSLRYAIQLITAASLVCRKRKGTEVQVDDIKRVYSLFLDESRSTQYMKEYQDAFLFNELKGETMDTS

SEQ ID NO: 5 - DNA, 1694 bp, Danio rerio
  CDS (1)...(1398)
atggcagcgcaggtggcaaccacaaaggttccggaggtgcgggacatcactcgcattgagagaatcggcgctcactctcacattcgtggccttgggttggatgatgctctggagccacgacaggtgtctcaggggatggtgggccagctggcatctcgaagggcagccgggctgattcttgaaatgatcaaagatggtcagattgctggtcgcgcagtccttattgctggccaacctgggacgggcaagacagctattgctatgggtattgcccagtcgcttggtcctgacacacctttcactgctctggctgggagtgagattttctctctggagatgagcaagactgaggctttgagccaagcattccgcaaggccataggagtcagaataaaagaggagacggagatcattgaaggagaggtggtagagattcagattgacagacctgctacaggaacaggtgcaaaagttggtaaactcactctgaagacgacagagatggaaaccatatatgatctggggaccaagatgatcgagagcctcagtaaagagagagtacaagccggtttctgcagagatgtgattactattgataaagccacagggaagattagcaagcttggacggtcatttactcgagcaagagactatgatgccatgggagcacagacgcagtttgtgcagtgtccagagggggaacttcaaaagaggaaggaggttgttcacaccgtctctcttcatgaaattgacgtcatcaacagccgcacacagggattcttggccttattttcaggcgacacaggagagattaagtcagaggttcgggagcagatcaatgctaaagtgtctgaatggagagaggaaggcaaagctgaaattattcctggggttctcttcatagacgaggtccacatgctggacatagaatgcttttcattcctgaaccgagccctagagagtgatctttctcctgtactcatcatggctaccaacagaggcatcactcgaattagaggcaccaattaccagagtcctcatggtattcccatagacatgctggaccgtctgcttatcatcgctactactccttacacagagaaagagaccagacagatcctaaagattcgctgtgaggaggaggatgttgagctcagtgaagaggcacatacagttttaacacgtatcggacaggagacttcactccgctacgccatccagctcatcagtactgcaggcctggtgtgccgtaaacgcaggggcacagaagtccaagtggaggacattaaaagggtttattctctcttcctggatgaggccaggtcctcacagtacatgaaagagtatcaggactccttcctcttcaatgaaacacaaacctctcagatggacacctcgtaacgagtggagctgtgaataagcatgtccagtcattgctcctgtgaagacctttctcttatgacctctcctagtgctgagccatctctcaagtgttcaggcaattcagtgttcatgcactcaaatcctcatcttcaagtcctttttttttttttttttttgctattgctagattttatatgttttgcaatgaataaaaataatttcttcttgtttggctgtataacatggcaacttaaaggatgaaataaaagatatttgcccataaaaaaaaaaaaaaaaaaaaaaaaaaaaaa

SEQ ID NO: 6 - protein, 466 aa, Danio rerio
MAAQVATTKVPEVRDITRIERIGAHSHIRGLGLDDALEPRQVSQGMVGQLASRRAAGLILEMIKDGQIAGRAVLIAGQPGTGKTAIAMGIAQSLGPDTPFTALAGSEIFSLEMSKTEALSQAFRKAIGVRIKEETEIIEGEVVEIQIDRPATGTGAKVGKLTLKTTEMETIYDLGTKMIESLSKERVQAGFCRDVITIDKATGKISKLGRSFTRARDYDAMGAQTQFVQCPEGELQKRKEVVHTVSLHEIDVINSRTQGFLALFSGDTGEIKSEVREQINAKVSEWREEGKAEIIPGVLFIDEVHMLDIECFSFLNRALESDLSPVLIMATNRGITRIRGTNYQSPHGIPIDMLDRLLIIATTPYTEKETRQILKIRCEEEDVELSEEAHTVLTRIGQETSLRYAIQLISTAGLVCRKRRGTEVQVEDIKRVYSLFLDEARSSQYMKEYQDSFLFNETQTSQMDTS

(19) United States
(12) Patent Application Publication
(10) Pub. No.: US 2005/0053935 A1
(43) Pub. Date: Mar. 10, 2005

(54) Methods for diagnosing and treating diseases and conditions of the heart or digestive system, and cancer

(76) Inventors: Mark C. Fishman, Newton Center, MA (US); Wolfgang Rottbauer, Regensburg (DE)

Correspondence Address: CLARK & ELBING LLP, 101 FEDERAL STREET, BOSTON, MA, 02110, US

(21) Appl. No.: 10/490,913

(22) PCT Filed: Oct. 1, 2002
(86) PCT No.: PCT/US02/31234

Related U.S. Application Data
(60) Provisional application No. 60/326,646, filed on Oct. 1, 2001.

(51) Int. Cl. C12Q 1/68

(52) U.S. Cl. 435/6

(57) ABSTRACT

The invention provides methods of diagnosing and treating diseases and conditions of the heart or digestive system and cancer, methods for identifying compounds that can be used to treat or to prevent such diseases and conditions, and methods of using these compounds to treat or to prevent such diseases and conditions. Also provided in the invention are animal model systems that can be used in screening methods.

### FIELD OF THE INVENTION

This invention relates to methods for diagnosing and treating diseases and conditions of the heart or digestive system, and cancer.

### BACKGROUND OF THE INVENTION

Heart disease is a general term used to describe many different heart conditions. For example, coronary artery disease, which is the most common heart disease, is characterized by constriction or narrowing of the arteries supplying the heart with oxygen-rich blood, and can lead to myocardial infarction, which is the death of a portion of the heart muscle. Heart failure is a condition resulting from the inability of the heart to pump an adequate amount of blood through the body. Heart failure is not a sudden, abrupt stop of heart activity but, rather, typically develops slowly over many years, as the heart gradually loses its ability to pump blood efficiently. Risk factors for heart failure include coronary artery disease, hypertension, valvular heart disease, cardiomyopathy, disease of the heart muscle, obesity, diabetes, and a family history of heart failure.

The zebrafish, Danio rerio, is a convenient organism to use in genetic and biochemical analyses of development. It has an accessible and transparent embryo, allowing direct observation of organ function from the earliest stages of development, has a short generation time, and is fecund.

### SUMMARY OF THE INVENTION

The invention provides diagnostic, drug screening, and therapeutic methods that are based on our observation that a mutation, designated fvo39-K, in the zebrafish reptin gene leads to a phenotype in zebrafish that is characterized by abnormal digestive system and heart development and function.

In a first aspect, the invention provides a method of determining whether a test subject (e.g., a mammal, such as a human) has or is at risk of developing a disease or condition related to reptin (e.g., a disease or condition of the digestive system or heart (e.g., heart failure), or cancer). This method involves analyzing a nucleic acid molecule of a sample from the test subject to determine whether the test subject has a mutation (e.g., the fvo39-K mutation; see below) in a gene encoding reptin. The presence of such a mutation indicates that the test subject has or is at risk of developing a disease related to reptin. This method can also involve the step of using nucleic acid molecule primers specific for a gene encoding reptin for nucleic acid molecule amplification of the gene by the polymerase chain reaction. It can further involve sequencing a nucleic acid molecule encoding reptin from a test subject.

In a second aspect, the invention provides a method for identifying compounds that can be used to treat or prevent a disease or condition associated with reptin, such as a disease or condition of the digestive system or heart, or cancer. This method involves contacting an organism (e.g., a zebrafish) having a mutation in a reptin gene (e.g., the fvo39-K mutation), and having a phenotype characteristic of such a disease or condition, with the compound, and determining the effect of the compound on the phenotype. Detection of an improvement in the phenotype indicates the identification of a compound that can be used to treat or prevent the disease or condition.

In a third aspect, the invention provides a method of treating or preventing a disease or condition (e.g., a disease or condition of the digestive system or heart, or cancer) related to reptin in a patient (e.g., a patient having a mutation (e.g., the fvo39-K mutation) in a reptin gene), involving administering to the patient a compound identified using the method described above. Also included in the invention is the use of such compounds in the treatment or prevention of such diseases or conditions, as well as the use of these compounds in the preparation of medicaments for such treatment or prevention.

In a fourth aspect, the invention provides an additional method of treating or preventing a disease or condition related to reptin in a patient, for example, a disease or condition of the digestive system or heart, or cancer. This method involves administering to the patient a functional reptin protein or a nucleic acid molecule (in, e.g., an expression vector) encoding the protein. Also included in the invention is the use of such proteins or nucleic acid molecules in the treatment or prevention of such diseases or conditions, as well as the use of these proteins or nucleic acid molecules in the preparation of medicaments for such treatment or prevention.

In a fifth aspect, the invention includes a substantially pure zebrafish reptin polypeptide. This polypeptide can include or consist essentially of, for example, an amino acid sequence that is substantially identical to the amino acid sequence of SEQ ID NO:2. The invention also includes variants of these polypeptides that include sequences that are at least 75%, 85%, 90%, or 95% (e.g., at least 96%, 97%, 98%, or 99%) identical to the sequences of these polypeptides, and which have reptin activity (as determined by effect on development or by, for example, analysis of ATPase and/or helicase activity; see below) or otherwise are characteristic of the diseases and conditions mentioned elsewhere herein. Fragments of these polypeptides are also included in the invention. For example, fragments that include any of the different domains of reptin, in varying combinations, are included. Preferably, reptin polypeptides according to the invention include Walker conserved motifs A and B (GQPGTGKT and DEVH, respectively; see SEQ ID NOs:2, 4, and 6). In addition, these polypeptides can have single-stranded DNA-dependent ATPase and/or ATP-dependent DNA helicase activity.

In a sixth aspect, the invention provides an isolated nucleic acid molecule (e.g., a DNA molecule) including a sequence encoding a zebrafish reptin polypeptide. This nucleic acid molecule can encode a polypeptide including or consisting essentially of an amino sequence that is substantially identical to the amino acid sequence of SEQ ID NO:2. The invention also includes nucleic acid molecules that hybridize to the complement of SEQ ID NO:1 under highly stringent conditions and encode polypeptides that have reptin activity or otherwise are characteristic of the diseases and conditions mentioned elsewhere herein.

In a seventh aspect, the invention provides a vector including the nucleic acid molecule described above.

In an eighth aspect, the invention includes a cell including the vector described above.

In a ninth aspect, the invention provides a non-human transgenic animal (e.g., a zebrafish or a mouse) including the nucleic acid molecule described above.

In a tenth aspect, the invention provides a non-human animal having a knockout mutation in one or both alleles encoding a reptin polypeptide.

In an eleventh aspect, the invention includes a cell from the non-human knockout animal described above.

In a twelfth aspect, the invention includes a non-human transgenic animal (e.g., a zebrafish) including a nucleic acid molecule encoding a mutant reptin polypeptide, e.g., a polypeptide having the fvo39-K mutation (see SEQ ID NOs:5 and 6, and below).

In a thirteenth aspect, the invention provides an antibody that specifically binds to a reptin polypeptide.

In a fourteenth aspect, the invention provides a method of modulating the activity of a reptin polypeptide in a patient, by administering to the patient an RNA that stimulates or inhibits this activity.

By “polypeptide” or “polypeptide fragment” is meant a chain of two or more (e.g., 10, 15, 20, 30, 50, 100, or 200, or more) amino acids, regardless of any post-translational modification (e.g., glycosylation or phosphorylation), constituting all or part of a naturally or non-naturally occurring polypeptide. By “post-translational modification” is meant any change to a polypeptide or polypeptide fragment during or after synthesis. Post-translational modifications can be produced naturally (such as during synthesis within a cell) or generated artificially (such as by recombinant or chemical means). A “protein” can be made up of one or more polypeptides.

By “reptin protein” or “reptin polypeptide” is meant a polypeptide that has at least 45%, preferably at least 60%, more preferably at least 75%, and most preferably at least 90% (e.g., at least 91%, 92%, 93%, 94%, 95%, 96%, 97%, 98%, or 99%) amino acid sequence identity to the sequence of a human (SEQ ID NO:4) or a zebrafish (SEQ ID NO:2) reptin polypeptide. A reptin polypeptide as defined herein plays a role in heart development, modeling, and function. It can be used as a marker of diseases and conditions associated with reptin, such as a disease or condition of the digestive system, heart disease (e.g., heart failure), or cancer (see below).

Polypeptide products from splice variants of reptin gene sequences and reptin genes containing mutations are also included in the invention. The splice variants can, for example, result in the insertion of short stretches of amino acids (e.g., 1, 2, 3, 4, 5, 6, 7, 8, 9, or 10 amino acids) at, e.g., a splice site, as is the case for the fvo39-k mutation (see below). Additional types of mutations include, for example, mutations that result in the introduction of a stop codon into the protein, resulting in the formation of a truncated protein.

By a “reptin nucleic acid molecule” is meant a nucleic acid molecule, such as a genomic DNA, cDNA, or RNA (e.g., mRNA) molecule, that encodes a reptin protein (e.g., a human (encoded by SEQ ID NO:3) or a zebrafish (encoded by SEQ ID NO:1) reptin protein), a reptin polypeptide, or a portion thereof, as defined above. A mutation in a reptin nucleic acid molecule can be characterized, for example, by the insertion of a premature stop codon anywhere in the reptin gene, or by a mutation in a splice donor site or intronic sequence, which leads to aberrant transcript production (e.g., transcripts with premature stop codons). In addition to fvo39-K and similar mutations, the invention includes any mutation that results in aberrant reptin protein production or function, including, only as examples, null mutations and additional mutations causing truncations.

The term “identity” is used herein to describe the relationship of the sequence of a particular nucleic acid molecule or polypeptide to the sequence of a reference molecule of the same type. For example, if a polypeptide or a nucleic acid molecule has the same amino acid or nucleotide residue at a given position, compared to a reference molecule to which it is aligned, there is said to be “identity” at that position. The level of sequence identity of a nucleic acid molecule or a polypeptide to a reference molecule is typically measured using sequence analysis software with the default parameters specified therein, such as the introduction of gaps to achieve an optimal alignment (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). These software programs match identical or similar sequences by assigning degrees of identity to various substitutions, deletions, or other modifications. Conservative substitutions typically include substitutions within the following groups: glycine, alanine, valine, isoleucine, and leucine; aspartic acid, glutamic acid, asparagine, and glutamine; serine and threonine; lysine and arginine; and phenylalanine and tyrosine.

A nucleic acid molecule or polypeptide is said to be “substantially identical” to a reference molecule if it exhibits, over its entire length, at least 51%, preferably at least 55%, 60%, or 65%, and most preferably 75%, 85%, 90%, or 95% identity to the sequence of the reference molecule. For polypeptides, the length of comparison sequences is at least 16 amino acids, preferably at least 20 amino acids, more preferably at least 25 amino acids, and most preferably at least 35 amino acids. For nucleic acid molecules, the length of comparison sequences is at least 50 nucleotides, preferably at least 60 nucleotides, more preferably at least 75 nucleotides, and most preferably at least 110 nucleotides. Of course, the length of comparison can be any length up to and including full length.

A reptin nucleic acid molecule or a reptin polypeptide is “analyzed” or subject to “analysis” if a test procedure is carried out on it that allows the determination of its biological activity or whether it is wild type or mutated. For example, one can analyze the reptin genes of an animal (e.g., a human or a zebrafish) by amplifying genomic DNA of the animal using the polymerase chain reaction, and then determining whether the amplified DNA contains a mutation, for example, the fvo39-K mutation, by, e.g., nucleotide sequence or restriction fragment analysis.

By “probe” or “primer” is meant a single-stranded DNA or RNA molecule of defined sequence that can base pair to a second DNA or RNA molecule that contains a complementary sequence (a “target”). The stability of the resulting hybrid depends upon the extent of the base pairing that occurs. This stability is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are well known to those skilled in the art. Probes or primers specific for reptin nucleic acid molecules, preferably, have greater than 45% sequence identity, more preferably at least 55-75% sequence identity, still more preferably at least 75-85% sequence identity, yet more preferably at least 85-99% sequence identity, and most preferably 100% sequence identity to the sequences of human (SEQ ID NO:3) or zebrafish (SEQ ID NO:1) reptin genes.

Probes can be detectably labeled, either radioactively or non-radioactively, by methods that are well known to those skilled in the art. Probes can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, northern hybridization, in situ hybridization, electrophoretic mobility shift assay (EMSA), and other methods that are well known to those skilled in the art.

A molecule, e.g., an oligonucleotide probe or primer, a gene or fragment thereof, a cDNA molecule, a polypeptide, or an antibody, can be said to be “detectably-labeled” if it is marked in such a way that its presence can be directly identified in a sample. Methods for detectably labeling molecules are well known in the art and include, without limitation, radioactive labeling (e.g., with an isotope, such as ³²P or ³⁵S) and nonradioactive labeling (e.g., with a fluorescent label, such as fluorescein).

By a “substantially pure polypeptide” is meant a polypeptide (or a fragment thereof) that has been separated from proteins and organic molecules that naturally accompany it. Typically, a polypeptide is substantially pure when it is at least 60%, by weight, free from the proteins and naturally occurring organic molecules with which it is naturally associated. Preferably, the polypeptide is a reptin polypeptide that is at least 75%, more preferably at least 90%, and most preferably at least 99%, by weight, pure. A substantially pure reptin polypeptide can be obtained, for example, by extraction from a natural source, by expression of a recombinant nucleic acid molecule encoding a reptin polypeptide, or by chemical synthesis. Purity can be measured by any appropriate method, e.g., by column chromatography, polyacrylamide gel electrophoresis, or HPLC analysis.

A polypeptide is substantially free of naturally associated components when it is separated from those proteins and organic molecules that accompany it in its natural state. Thus, a protein that is chemically synthesized or produced in a cellular system that is different from the cell in which it is naturally produced is substantially free from its naturally associated components. Accordingly, substantially pure polypeptides not only include those that are derived from eukaryotic organisms, but also those synthesized in E. coli, other prokaryotes, or in other such systems.

By “isolated nucleic acid molecule” is meant a nucleic acid molecule that is removed from the environment in which it naturally occurs. For example, a naturally-occurring nucleic acid molecule present in the genome of cell or as part of a gene bank is not isolated, but the same molecule, separated from the remaining part of the genome, as a result of, e.g., a cloning event (amplification), is “isolated.” Typically, an isolated nucleic acid molecule is free from nucleic acid regions (e.g., coding regions) with which it is immediately contiguous, at the 5′ or 3′ ends, in the naturally occurring genome. Such isolated nucleic acid molecules can be part of a vector or a composition and still be isolated, as such a vector or composition is not part of its natural environment.

An antibody is said to “specifically bind” to a polypeptide if it recognizes and binds to the polypeptide (e.g., a reptin polypeptide), but does not substantially recognize and bind to other molecules (e.g., non-reptin-related polypeptides) in a sample, e.g., a biological sample, which naturally includes the polypeptide.

By “high stringency conditions” is meant conditions that allow hybridization comparable with the hybridization that occurs using a DNA probe of at least 100, e.g., 200, 350, or 500, nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1× Denhardt's solution, 10% dextran sulfate, and 0.1% SDS, at a temperature of 42° C. (These are typical conditions for high stringency northern or Southern hybridizations.) High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually 16 nucleotides or longer for PCR or sequencing, and 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

By “sample” is meant a tissue biopsy, amniotic fluid, cell, blood, serum, urine, stool, or other specimen obtained from a patient or a test subject. The sample can be analyzed to detect a mutation in a reptin gene, or expression levels of a reptin gene, by methods that are known in the art. For example, methods such as sequencing, single-strand conformational polymorphism (SSCP) analysis, or restriction fragment length polymorphism (RFLP) analysis of PCR products derived from a patient sample can be used to detect a mutation in a reptin gene; ELISA and other immunoassays can be used to measure levels of a reptin polypeptide; and PCR can be used to measure the level of a Reptin nucleic acid molecule.

By “reptin-related disease” or “reptin-related condition” is meant a disease or condition that results from inappropriately high or low expression of a reptin gene, or a mutation in a reptin gene (including control sequences, such as promoters) that alters the biological activity of a reptin nucleic acid molecule or polypeptide. Reptin-related diseases and conditions can arise in any tissue in which reptin is expressed during prenatal or post-natal life. Reptin-related diseases and conditions can include diseases or conditions of the digestive system or heart (e.g., heart failure), or cancer (see below).

The invention provides several advantages. For example, using the diagnostic methods of the invention it is possible to detect an increased likelihood of diseases or conditions associated with reptin, such as diseases of the digestive system or heart, or cancer, in a patient, so that appropriate intervention can be instituted before any symptoms occur. This may be useful, for example, with patients in high-risk groups for such diseases or conditions. Also, the diagnostic methods of the invention facilitate determination of the etiology of such an existing disease or condition in a patient, so that an appropriate approach to treatment can be selected. In addition, the screening methods of the invention can be used to identify compounds that can be used to treat or to prevent these diseases or conditions. The invention can also be used to treat diseases or conditions (e.g., organ failure) for which, prior to the invention, the only treatment was organ transplantation, which is limited by the availability of donor organs and the possibility of organ rejection.

Other features and advantages of the invention will be apparent from the following detailed description, the drawings, and the claims.

### BRIEF DESCRIPTION OF THE DRAWINGS

FIG. 1 is an integrated genetic and physical map of the zebrafish fvo39-K region.

FIG. 2 is a schematic representation of the genomic structure of the zebrafish reptin gene, which contains 15 exons.

FIG. 3 is an amino acid sequence alignment of zebrafish, xenopus, human, drosophila, and yeast reptins.

FIG. 4 is a schematic illustration of the effects of the intronic point mutation in fvo39-K.

FIG. 5 is a schematic representation of the cDNA and amino acid sequences of zebrafish (SEQ ID NOs:1 and 2, respectively) and human (SEQ ID NOs:3 and 4, respectively) wild type reptin. Also provided in FIG. 5 are the cDNA and amino acid sequences of the zebrafish fvo39-K mutant (SEQ ID NOs:5 and 6, respectively).

### DETAILED DESCRIPTION

The invention provides methods of diagnosing, preventing, and treating diseases and conditions associated with reptin, such as diseases or conditions of the digestive system or heart, or cancer, and screening methods for identifying compounds that can be used to treat or prevent such diseases and conditions. In particular, we have identified a genetic mutation, fvo39-K, which perturbs development and function of the digestive system and heart. We show here that the fvo39-K gene encodes reptin.

The diagnostic methods of the invention thus involve detection of mutations in genes encoding reptin proteins, while the compound identification methods involve screening for compounds that affect the phenotype of organisms having mutations in genes encoding reptin or other models of appropriate diseases and conditions. Compounds identified in this manner, as well as reptin genes, proteins, and antibodies themselves, can be used in methods to treat or prevent diseases and conditions associated with reptin (see below).

The invention also provides animal model systems (e.g., zebrafish having mutations (e.g., the fvo39-K mutation) in reptin genes, or mice (or other animals) having such mutations) that can be used in the screening methods mentioned above, as well as the reptin protein, and genes encoding this protein. Also included in the invention are genes encoding mutant zebrafish reptin proteins (e.g., genes having the fvo39-K mutation) and proteins encoded by these genes. Antibodies that specifically bind to these proteins (wild type or mutant) are also included in the invention.

The diagnostic, screening, and therapeutic methods of the invention, as well as the animal model systems, proteins, and genes of the invention, are described further, as follows, after a brief description of diseases and conditions associated with reptin, which can be diagnosed, prevented, or treated according to the invention.

Reptin-Associated Diseases or Conditions

Abnormalities in reptin genes or proteins can be associated with any of a wide variety of diseases or conditions, all of which can thus be diagnosed, prevented, or treated using the methods of the invention. For example, as discussed above, the fvo39-K mutation in zebrafish is characterized by abnormal digestive system and heart development and function. Thus, detection of abnormalities in reptin genes or their expression can be used in methods to diagnose, or to monitor the treatment or development of, diseases or conditions of the digestive system or the heart (see below). We have also found that reptin plays a role in cell growth control. Thus, detection of abnormalities in this gene (or the protein it encodes) can be used in the diagnosis of cancer, as well in monitoring cancer treatment. In addition, compounds that are identified in the screening methods described herein, as well as reptin nucleic acid molecules, proteins, and antibodies themselves, can be used in methods to prevent or treat such diseases or conditions. The invention can also be applied in the context of eye disease.

An example of a disease or condition of the heart that can be diagnosed, prevented, or treated using the methods of the invention is heart failure. Examples of heart failure include congestive heart failure, which is characterized by fluid in the lungs or body, resulting from failure of the heart in acting as a pump; right sided heart failure (right ventricular), which is characterized by failure of the pumping action of the right ventricle, resulting in swelling of the body, especially the legs and abdomen; left sided heart failure (left ventricular), which is caused by failure of the pumping action of the left side of the heart, resulting in congestion of the lungs; forward heart failure, which is characterized by the inability of the heart to pump blood forward at a sufficient rate to meet the oxygen needs of the body at rest or during exercise; backward heart failure, which is characterized by the ability of the heart to meet the needs of the body only if heart filling pressures are abnormally high; low-output, which is characterized by failure to maintain blood output; and high-output, which is characterized by heart failure symptoms, even when cardiac output is high.

The methods described herein can also be used in the diagnosis, prevention, and treatment of cardiovascular diseases other than heart failure, such as coronary artery disease, heart fibrillation (e.g., atrial fibrillation), or conditions associated with valve formation defects, and, thus, detection of abnormalities in reptin genes or their expression can be used in methods to diagnose and monitor these conditions as well.

Diseases or conditions of the digestive tract that can be diagnosed (and prevented or treated) using the methods of the invention include any diseases or conditions that affect a digestive organ, such as the intestine (large or small), liver, biliary tract, pancreas, stomach, gall bladder, or esophagus. For example, the methods can be used to diagnose (or to treat) digestive organ failure (e.g., liver failure), inflammatory bowel disease (e.g., Crohn's disease or ulcerative colitis), diverticular disease (e.g., diverticulitis or diverticulosis), malabsorption, steatorrhea, ischemic bowel disease, irritable bowel syndrome, celiac disease, colitis, hepatitis (e.g., autoimmune hepatitis or hepatitis A, B, C, D, E, or G), cirrhosis, fatty liver, gastritis (acute and chronic), gastric ulcer, hyperplastic gastropathy, peptic ulcer, oral-pharyngeal dysphagia, achalasia, and gastro-esophageal reflux disease.

The methods of the invention can also be used in the diagnosis (and prevention or treatment) of cancer, e.g., cancer of the digestive tract. For example, the methods of the invention can be used to diagnose or to treat colon cancer, rectal cancer, liver cancer (e.g., hepatocellular carcinoma), pancreatic cancer (exocrine or islet), cancer of the gall bladder, esophageal cancer, stomach cancer, or bile duct cancer, and these cancers can be, for example, adenocarcinomas, adenomas, carcinoids, or carcinomas.

Diagnostic Methods

Nucleic acid molecules encoding reptin proteins, as well as polypeptides encoded by these nucleic acid molecules and antibodies specific for these polypeptides, can be used in methods to diagnose or to monitor diseases and conditions involving mutations in, or inappropriate expression of, genes encoding this protein.

The diagnostic methods of the invention can be used, for example, with patients that have a disease or condition associated with reptin, in an effort to determine its etiology and, thus, to facilitate selection of an appropriate course of treatment. The diagnostic methods can also be used with patients who have not yet developed, but who are at risk of developing, such a disease or condition, or with patients that are at an early stage of developing such a disease or condition. Also, the diagnostic methods of the invention can be used in prenatal genetic screening, for example, to identify parents who may be carriers of a recessive mutation in a gene encoding a reptin protein. In addition, the methods can be used to investigate whether a reptin mutation may be contributing to a disease or condition (e.g., heart disease) in a patient, by determining whether a reptin gene of a patient includes a mutation. The methods of the invention can be used to diagnose (or to prevent or treat) the disorders described herein in any mammal, for example, in humans, domestic pets, or livestock.

Abnormalities in reptin that can be detected using the diagnostic methods of the invention include those characterized by, for example, (i) a gene encoding a reptin protein containing a mutation that results in the production of an abnormal reptin protein, (ii) an abnormal reptin polypeptide itself (e.g., a truncated protein), and (iii) a mutation in a reptin gene that results in production of an abnormal amount of this protein. Detection of such abnormalities can be used to diagnose human diseases or conditions related to reptin, such as those affecting the digestive system or the heart. Exemplary of the mutations in reptin genes is the fvo39-K mutation, which is described further below.

A mutation in a reptin gene can be detected in any tissue of a subject, even one in which this protein is not expressed. Because of the possibly limited number of tissues in which these proteins may be expressed, for limited time periods, and because of the possible undesirability of sampling such tissues (e.g., heart tissue or digestive organs) for assays, it may be preferable to detect mutant genes in other, more easily obtained sample types, such as in blood or amniotic fluid samples.

Detection of a mutation in a gene encoding a reptin protein can be carried out using any standard diagnostic technique. For example, a biological sample obtained from a patient can be analyzed for one or more mutations (e.g., a fvo39-K mutation) in nucleic acid molecules encoding a reptin protein using a mismatch detection approach. Generally, this approach involves polymerase chain reaction (PCR) amplification of nucleic acid molecules from a patient sample, followed by identification of a mutation (i.e., a mismatch) by detection of altered hybridization, aberrant electrophoretic gel migration, binding, or cleavage mediated by mismatch binding proteins, or by direct nucleic acid molecule sequencing. Any of these techniques can be used to facilitate detection of a mutant gene encoding a reptin protein, and each is well known in the art. For instance, examples of these techniques are described by Orita et al. (Proc. Natl. Acad. Sci. U.S.A. 86:2766-2770, 1989) and Sheffield et al. (Proc. Natl. Acad. Sci. U.S.A. 86:232-236, 1989).

As noted above, in addition to facilitating diagnosis of an existing disease or condition, mutation detection assays also provide an opportunity to diagnose a predisposition to disease related to a mutation in a reptin gene before the onset of symptoms. For example, a patient who is heterozygous for a gene encoding an abnormal reptin protein (or an abnormal amount thereof) that suppresses normal reptin biological activity or expression may show no clinical symptoms of a disease related to such proteins, and yet possess a higher than normal probability of developing such disease. Given such a diagnosis, a patient can take precautions to minimize exposure to adverse environmental factors, and can carefully monitor their medical condition, for example, through frequent physical examinations. As mentioned above, this type of diagnostic approach can also be used to detect a mutation in a gene encoding the reptin protein in prenatal screens.

While it may be preferable to carry out diagnostic methods for detecting a mutation in a reptin gene using genomic DNA from readily accessible tissues, as noted above, mRNA encoding this protein, or the protein itself, can also be assayed from tissue samples in which it is expressed. Expression levels of a gene encoding reptin in such a tissue sample from a patient can be determined by using any of a number of standard techniques that are well known in the art, including northern blot analysis and quantitative PCR (see, e.g., Ausubel et al., supra; PCR Technology: Principles and Applications for DNA Amplification, H. A. Ehrlich, Ed., Stockton Press, NY; Yap et al. Nucl. Acids. Res. 19:4294, 1991).

In another diagnostic approach of the invention, an immunoassay is used to detect or to monitor the level of a reptin protein in a biological sample. Polyclonal or monoclonal antibodies specific for the reptin protein can be used in any standard immunoassay format (e.g., ELISA, Western blot, or RIA; see, e.g., Ausubel et al., supra) to measure polypeptide the levels of reptin. These levels can be compared to levels of reptin in a sample from an unaffected individual. Detection of a decrease in production of reptin using this method, for example, may be indicative of a condition or a predisposition to a condition involving insufficient biological activity of the reptin protein.

Immunohistochemical techniques can also be utilized for detection of reptin protein in patient samples. For example, a tissue sample can be obtained from a patient, sectioned, and stained for the presence of reptin using an anti-reptin antibody and any standard detection system (e.g., one that includes a secondary antibody conjugated to an enzyme, such as horseradish peroxidase). General guidance regarding such techniques can be found in, e.g., Bancroft et al., Theory and Practice of Histological Techniques, Churchill Livingstone, 1982, and Ausubel et al., supra.

Identification of Molecules that can be used to Treat or to Prevent Diseases or Conditions Associated with Reptin

Identification of a mutation in the gene encoding reptin as resulting in a phenotype that results in abnormal digestive system and heart development and function facilitates the identification of molecules (e.g., small organic or inorganic molecules, antibodies, peptides, or nucleic acid molecules) that can be used to treat or to prevent diseases or conditions associated with reptin, as discussed above. The effects of candidate compounds on such diseases or conditions can be investigated using, for example, the zebrafish system. As is mentioned above, the zebrafish, Danio rerio, is a convenient organism to use in the genetic analysis of development. It has an accessible and transparent embryo, allowing direct observation of organ function from the earliest stages of development, has a short generation time, and is fecund. As discussed further below, zebrafish and other organisms (e.g., mice, drosophila, and yeast) having a reptin mutation, such as the fvo39-K mutation, which can be used in these methods, are also included in the invention.

In one example of the screening methods of the invention, a zebrafish having a mutation in a gene encoding the reptin protein (e.g., a zebrafish having the fvo39-K mutation) is contacted with a candidate compound, and the effect of the compound on the development of an abnormal digestive system or heart, or on the status of such an existing abnormality, is monitored relative to an untreated, identically mutant control.

After a compound has been shown to have a desired effect in the zebrafish system, it can be tested in other models of digestive system or heart disease, for example, in mice or other animals having a mutation in a gene encoding reptin. Alternatively, testing in such animal model systems can be carried out in the absence of zebrafish testing.

Cell culture-based assays can also be used in the identification of molecules that increase or decrease reptin levels or biological activity. According to one approach, candidate molecules are added at varying concentrations to the culture medium of cells expressing reptin mRNA. Reptin biological activity is then measured using standard techniques. For example, the effect of a compound on development, as described herein, can be determined. Alternatively, helicase activity can be measured, using standard methods. The measurement of biological activity can include the measurement of reptin protein and nucleic acid molecule levels.

In general, novel drugs for the prevention or treatment of diseases related to mutations in genes encoding reptin can be identified from large libraries of natural products, synthetic (or semi-synthetic) extracts, and chemical libraries using methods that are well known in the art. Those skilled in the field of drug discovery and development will understand that the precise source of test extracts or compounds is not critical to the screening methods of the invention and that dereplication, or the elimination of replicates or repeats of materials already known for their therapeutic activities for reptin, can be employed whenever possible.

Candidate compounds to be tested include purified (or substantially purified) molecules or one or more components of a mixture of compounds (e.g., an extract or supernatant obtained from cells; Ausubel et al., supra), and such compounds further include both naturally occurring or artificially derived chemicals and modifications of existing compounds. For example, candidate compounds can be polypeptides, synthesized organic or inorganic molecules, naturally occurring organic or inorganic molecules, nucleic acid molecules, and components thereof.

Numerous sources of naturally occurring candidate compounds are readily available to those skilled in the art. For example, naturally occurring compounds can be found in cell (including plant, fungal, prokaryotic, and animal) extracts, mammalian serum, growth medium in which mammalian cells have been cultured, protein expression libraries, or fermentation broths. In addition, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). Furthermore, libraries of natural compounds can be produced, if desired, according to methods that are known in the art, e.g., by standard extraction and fractionation.

Artificially derived candidate compounds are also readily available to those skilled in the art. Numerous methods are available for generating random or directed synthesis (e.g., semi-synthesis or total synthesis) of any number of chemical compounds, including, for example, saccharide-, lipid-, peptide-, and nucleic acid molecule-based compounds. In addition, synthetic compound libraries are commercially available from Brandon Associates (Merrimack, N.H.) and Aldrich Chemicals (Milwaukee, Wis.). Libraries of synthetic compounds can also be produced, if desired, according to methods known in the art, e.g., by standard extraction and fractionation. Furthermore, if desired, any library or compound can be readily modified using standard chemical, physical, or biochemical methods. The techniques of modern synthetic chemistry, including combinatorial chemistry, can also be used (reviewed in Schreiber, Bioorganic and Medicinal Chemistry 6:1172-1152, 1998; Schreiber, Science 287:1964-1969, 2000).

When a crude extract is found to have an effect on the development or persistence of a reptin-associated disease, further fractionation of the positive lead extract can be carried out to isolate chemical constituents responsible for the observed effect. Thus, the goal of the extraction, fractionation, and purification process is the careful characterization and identification of a chemical entity within the crude extract having a desired activity. The same assays described herein for the detection of activities in mixtures of compounds can be used to purify the active component and to test derivatives of these compounds. Methods of fractionation and purification of such heterogeneous extracts are well known in the art. If desired, compounds shown to be useful agents for treatment can be chemically modified according to methods known in the art.

In general, compounds that are found to activate reptin expression or activity may be used in the prevention or treatment of diseases or conditions of the digestive system or heart, such as those listed above.

Animal Model Systems

The invention also provides animal model systems for use in carrying out the screening methods described above. Examples of these model systems include zebrafish and other animals, such as mice, that have a mutation (e.g., the fvo39-K mutation) in a reptin gene. For example, a zebrafish model that can be used in the invention can include a mutation that results in a lack of reptin protein production or production of a truncated (e.g., by introduction of a stop codon or a splice site mutation) or otherwise altered reptin gene product. As a specific example, a zebrafish having the fvo39-K mutation can be used (see below).

Treatment or Prevention of Reptin-Associated Diseases or Conditions

Compounds identified using the screening methods described above can be used to treat patients that have or are at risk of developing diseases or conditions of the digestive system or heart, or cancer. Nucleic acid molecules encoding the reptin protein, as well as these proteins themselves, can also be used in such methods.

Treatment may be required only for a short period of time or may, in some form, be required throughout a patient's lifetime. Any continued need for treatment, however, can be determined using, for example, the diagnostic methods described above. In considering various therapies, it is to be understood that such therapies are, preferably, targeted to the affected or potentially affected organ (e.g., a digestive organ or the heart). Such targeting can be achieved using standard methods.

Treatment or prevention of diseases resulting from a mutated reptin gene can be accomplished, for example, by modulating the function of a mutant reptin protein. Treatment can also be accomplished by delivering normal reptin protein to appropriate cells, altering the levels of normal or mutant reptin protein, replacing a mutant gene encoding a reptin protein with a normal gene encoding a reptin protein, or administering a normal gene encoding a reptin protein. It is also possible to correct the effects of a defect in a gene encoding a reptin protein by modifying the physiological pathway (e.g., a signal transduction pathway) in which a reptin protein participates.

In a patient diagnosed as being heterozygous for a gene encoding a mutant reptin protein, or as susceptible to such mutations or aberrant reptin expression (even if those mutations or expression patterns do not yet result in alterations in expression or biological activity of reptin), any of the therapies described herein can be administered before the occurrence of the disease phenotype. In particular, compounds shown to have an effect on the phenotype of mutants, or to modulate expression of reptin proteins, can be administered to patients diagnosed with potential or actual disease by any standard dosage and route of administration.

Any appropriate route of administration can be employed to administer a compound identified as described above, a reptin gene, protein, or antibody, according to the invention. For example, administration can be parenteral, intravenous, intra-arterial, subcutaneous, intramuscular, intraventricular, intracapsular, intraspinal, intracistemal, intraperitoneal, intranasal, by aerosol, by suppository, or oral.

A therapeutic compound of the invention can be administered within a pharmaceutically acceptable diluent, carrier, or excipient, in unit dosage form.

Administration can begin before or after the patient is symptomatic. Methods that are well known in the art for making formulations are found, for example, in Remington 's Pharmaceutical Sciences (18^{th} edition), ed. A. Gennaro, 1990, Mack Publishing Company, Easton, Pa. Therapeutic formulations can be in the form of liquid solutions or suspensions. Formulations for parenteral administration can contain, for example, excipients, sterile water, or saline; polyalkylene glycols, such as polyethylene glycol; oils of vegetable origin; or hydrogenated napthalenes. Biocompatible, biodegradable lactide polymer, lactide/glycolide copolymer, or polyoxyethylene-polyoxypropylene copolymers can be used to control the release of the compounds. Other potentially useful parenteral delivery systems include ethylene-vinyl acetate copolymer particles, osmotic pumps, implantable infusion systems, and liposomes. For oral administration, formulations can be in the form of tablets or capsules. Formulations for inhalation can contain excipients, for example, lactose, or can be aqueous solutions containing, for example, polyoxyethylene-9-lauryl ether, glycocholate, and deoxycholate, or can be oily solutions for administration in the form of nasal drops or as a gel. Alternatively, intranasal formulations can be in the form of powders or aerosols.

To replace a mutant protein with normal protein, or to add protein to cells that do not express a sufficient amount of reptin or normal reptin, it may be necessary to obtain large amounts of pure reptin protein from cell culture systems in which the protein is expressed (see, e.g., below). Delivery of the protein to the affected tissue can then be accomplished using appropriate packaging or administration systems.

Gene therapy is another therapeutic approach for preventing or ameliorating diseases caused by reptin gene defects. Nucleic acid molecules encoding wild type reptin protein can be delivered to cells that lack sufficient, normal reptin protein biological activity (e.g., cells carrying mutations (e.g., the fvo39-K mutation) in reptin genes). The nucleic acid molecules must be delivered to those cells in a form in which they can be taken up by the cells and so that sufficient levels of protein, to provide effective reptin protein function, can be produced. Alternatively, for some reptin mutations, it may be possible to slow the progression of the resulting disease or to modulate reptin protein activity by introducing another copy of a homologous gene bearing a second mutation in that gene, to alter the mutation, or to use another gene to block any negative effect.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, the full length reptin gene, or a portion thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of therapeutic DNA into cells predicted to be subject to diseases involving the reptin protein. For example, a reptin nucleic acid molecule or an antisense nucleic acid molecule can be introduced into a cell by lipofection (Felgner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990).

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal reptin protein into a cultivatable cell type ex vivo (e.g., an autologous or heterologous primary cell or progeny thereof), after which the cell (or its descendants) are injected into a targeted tissue.

Reptin cDNA expression for use in gene therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct reptin expression. The enhancers used can include, without limitation, those that are characterized as tissue- or cell-specific enhancers. Alternatively, if a reptin genomic clone is used as a therapeutic construct (such clones can be identified by hybridization with reptin cDNA, as described herein), regulation can be mediated by the cognate regulatory sequences or, if desired, by regulatory sequences derived from a heterologous source, including any of the promoters or regulatory elements described above.

Molecules for effecting antisense-based strategies can be employed to explore reptin protein gene function, as a basis for therapeutic drug design, as well as to treat reptin-associated diseases. These strategies are based on the principle that sequence-specific suppression of gene expression (via transcription or translation) can be achieved by intracellular hybridization between genomic DNA or mRNA and a complementary antisense species. The formation of a hybrid RNA duplex interferes with transcription of the target reptin-encoding genomic DNA molecule, or processing, transport, translation, or stability of the target reptin mRNA molecule.

Antisense strategies can be delivered by a variety of approaches. For example, antisense oligonucleotides or antisense RNA can be directly administered (e.g., by intravenous injection) to a subject in a form that allows uptake into cells. Alternatively, viral or plasmid vectors that encode antisense RNA (or antisense RNA fragments) can be introduced into a cell in vivo or ex vivo. Antisense effects can be induced by control (sense) sequences; however, the extent of phenotypic changes is highly variable. Phenotypic effects induced by antisense molecules are based on changes in criteria such as protein levels, protein activity measurement, and target mRNA levels.

Reptin gene therapy can also be accomplished by direct administration of antisense reptin mRNA to a cell that is expected to be adversely affected by the expression of wild type or mutant reptin protein. The antisense reptin mRNA can be produced and isolated by any standard technique, but is most readily produced by in vitro transcription using an antisense reptin cDNA under the control of a high efficiency promoter (e.g., the T7 promoter). Administration of antisense reptin mRNA to cells can be carried out by any of the methods for direct nucleic acid molecule administration described above.

An alternative strategy for inhibiting reptin protein function using gene therapy involves intracellular expression of an anti-reptin protein antibody or a portion of an anti-reptin protein antibody. For example, the gene (or gene fragment) encoding a monoclonal antibody that specifically binds to a reptin protein and inhibits its biological activity can be placed under the transcriptional control of a tissue-specific gene regulatory sequence.

Another therapeutic approach included in the invention involves administration of a recombinant reptin polypeptide, either directly to the site of a potential or actual disease-affected tissue (for example, by injection) or systemically (for example, by any conventional recombinant protein administration technique). The dosage of the reptin protein depends on a number of factors, including the size and health of the individual patient but, generally, between 0.001 mg and 100 mg, inclusive, is administered per day to an adult in any pharmaceutically acceptable formulation.

In addition to the therapeutic methods described herein, involving administration of reptin-modulating compounds, reptin proteins, or reptin nucleic acids to patients, the invention provides methods of culturing organs in the presence of such molecules. In particular, as is noted above, a reptin mutation is associated with abnormal digestive system and heart development and function. Thus, culturing a digestive system organ or heart tissue in the presence of these molecules can be used to promote their proper development or function. This tissue can be that which is being prepared for transplant from, e.g., an allogeneic or xenogeneic donor, as well as synthetic tissue or organs.

Synthesis of Reptin Proteins, Polypeptides, and Polvyeptide Fragments

Those skilled in the art of molecular biology will understand that a wide variety of expression systems can be used to produce recombinant reptin proteins. As discussed further below, the precise host cell used is not critical to the invention. The reptin proteins can be produced in a prokaryotic host (e.g., E. coli) or in a eukaryotic host (e.g., S. cerevisiae, insect cells, such as Sf9 cells, or mammalian cells, such as COS-1, NIH 3T3, or HeLa cells). These cells are commercially available from, for example, the American Type Culture Collection, Manassas, Va. (see also Ausubel et al., supra). The method of transformation and the choice of expression vehicle (e.g., expression vector) will depend on the host system selected. Transformation and transfection methods are described, e.g., in Ausubel et al., supra, and expression vehicles can be chosen from those provided, e.g., in Pouwels et al., Cloning Vectors: A Laboratory Manual, 1985, Supp. 1987. Specific examples of expression systems that can be used in the invention are described further as follows.

For protein expression, eukaryotic or prokaryotic expression systems can be generated in which reptin gene sequences are introduced into a plasmid or other vector, which is then used to transform living cells. Constructs in which full-length reptin cDNAs, containing the entire open reading frame, inserted in the correct orientation into an expression plasmid, can be used for protein expression. Alternatively, portions of reptin gene sequences, including wild type or mutant reptin sequences, can be inserted. Prokaryotic and eukaryotic expression systems allow various important functional domains of reptin proteins to be recovered, if desired, as fusion proteins, and then used for binding, structural, and functional studies, and also for the generation of antibodies.

Typical expression vectors contain promoters that direct synthesis of large amounts of mRNA corresponding to a nucleic acid molecule that has been inserted into the vector. They can also include a eukaryotic or prokaryotic origin of replication, allowing for autonomous replication within a host cell, sequences that confer resistance to an otherwise toxic drug, thus allowing vector-containing cells to be selected in the presence of the drug, and sequences that increase the efficiency with which the synthesized mRNA is translated. Stable, long-term vectors can be maintained as freely replicating entities by using regulatory elements of, for example, viruses (e.g., the OriP sequences from the Epstein Barr Virus genome). Cell lines can also be produced that have the vector integrated into genomic DNA of the cells and, in this manner, the gene product can be produced in the cells on a continuous basis.

Expression of foreign molecules in bacteria, such as Escherichia coli, requires the insertion of a foreign nucleic acid molecule, e.g., a reptin nucleic acid molecule, into a bacterial expression vector. Such plasmid vectors include several elements required for the propagation of the plasmid in bacteria, and for expression of foreign DNA contained within the plasmid. Propagation of only plasmid-bearing bacteria is achieved by introducing, into the plasmid, a selectable marker-encoding gene that allows plasmid-bearing bacteria to grow in the presence of an otherwise toxic drug. The plasmid also contains a transcriptional promoter capable of directing synthesis of large amounts of mRNA from the foreign DNA. Such promoters can be, but are not necessarily, inducible promoters that initiate transcription upon induction by culture under appropriate conditions (e.g., in the presence of a drug that activates the promoter). The plasmid also, preferably, contains a polylinker to simplify insertion of the gene in the correct orientation within the vector.

Once an appropriate expression vector containing a reptin gene, or a fragment, fusion, or mutant thereof, is constructed, it can be introduced into an appropriate host cell using a transformation technique, such as, for example, calcium phosphate transfection, DEAE-dextran transfection, electroporation, microinjection, protoplast fusion, or liposome-mediated transfection. Host cells that can be transfected with the vectors of the invention can include, but are not limited to, E. coli or other bacteria, yeast, fungi, insect cells (using, for example, baculoviral vectors for expression), or cells derived from mice, humans, or other animals. Mammalian cells can also be used to express reptin proteins using a virus expression system (e.g., a vaccinia virus expression system) described, for example, in Ausubel et al., supra.

In vitro expression of reptin proteins, fusions, polypeptide fragments, or mutants encoded by cloned DNA can also be carried out using the T7 late-promoter expression system. This system depends on the regulated expression of T7 RNA polymerase, an enzyme encoded in the DNA of bacteriophage T7. The T7 RNA polymerase initiates transcription at a specific 23 base pair promoter sequence called the T7 late promoter. Copies of the T7 late promoter are located at several sites on the T7 genome, but none are present in E. coli chromosomal DNA. As a result, in T7-infected E. coli, T7 RNA polymerase catalyzes transcription of viral genes, but not E. coli genes. In this expression system, recombinant E. coli cells are first engineered to carry the gene encoding T7 RNA polymerase next to the lac promoter. In the presence of IPTG, these cells transcribe the T7 polymerase gene at a high rate and synthesize abundant amounts of T7 RNA polymerase. These cells are then transformed with plasmid vectors that carry a copy of the T7 late promoter protein. When IPTG is added to the culture medium containing these transformed E. coli cells, large amounts of T7 RNA polymerase are produced. The polymerase then binds to the T7 late promoter on the plasmid expression vectors, catalyzing transcription of the inserted cDNA at a high rate. Since each E. coli cell contains many copies of the expression vector, large amounts of mRNA corresponding to the cloned cDNA can be produced in this system and the resulting protein can be radioactively labeled.

Plasmid vectors containing late promoters and the corresponding RNA polymerases from related bacteriophages, such as T3, T5, and SP6, can also be used for in vitro production of proteins from cloned DNA. E. coli can also be used for expression using an M13 phage, such as mGPI-2. Furthermore, vectors that contain phage lambda regulatory sequences, or vectors that direct the expression of fusion proteins, for example, a maltose-binding protein fusion protein or a glutathione-S-transferase fusion protein, also can be used for expression in E. coli.

Eukaryotic expression systems are useful for obtaining appropriate post-translational modification of expressed proteins. Transient transfection of a eukaryotic expression plasmid containing a reptin gene into a eukaryotic host cell allows the transient production of a reptin protein by the transfected host cell. Reptin proteins can also be produced by a stably-transfected eukaryotic (e.g., mammalian) cell line. A number of vectors suitable for stable transfection of mammalian cells are available to the public (see, e.g., Pouwels et al., supra), as are methods for constructing lines including such cells (see, e.g., Ausubel et al., supra).

In one example, cDNA encoding a reptin protein, fusion, mutant, or polypeptide fragment is cloned into an expression vector that includes the dihydrofolate reductase (DFR) gene. Integration of the plasmid and, therefore, integration of the fvo39-K protein-encoding gene, into the host cell chromosome is selected for by inclusion of 0.01-300 μM methotrexate in the cell culture medium (Ausubel et al., supra). This dominant selection can be accomplished in most cell types. Recombinant protein expression can be increased by DHFR-mediated amplification of the transfected gene. Methods for selecting cell lines bearing gene amplifications are described in Ausubel et al., supra. These methods generally involve extended culture in medium containing gradually increasing levels of methotrexate. The most commonly used DHFR-containing expression vectors are pCVSEII-DHFR and pAdD26SV(A) (described, for example, in Ausubel et al., supra). The host cells described above or, preferably, a DHFR-deficient CHO cell line (e.g., CHO DHFR-cells, ATCC Accession No. CRL 9096) are among those that are most preferred for DHFR selection of a stably transfected cell line or DHFR-mediated gene amplification.

Another preferred eukaryotic expression system is the baculovirus system using, for example, the vector pBacPAK9, which is available from Clontech (Palo Alto, Calif.). If desired, this system can be used in conjunction with other protein expression techniques, for example, the myc tag approach described by Evan et al. (Molecular and Cellular Biology 5:3610-3616, 1985).

Once a recombinant protein is expressed, it can be isolated from the expressing cells by cell lysis followed by protein purification techniques, such as affinity chromatography. In this example, an anti-reptin antibody, which can be produced by the methods described herein, can be attached to a column and used to isolate the recombinant reptin. Lysis and fractionation of reptin-harboring cells prior to affinity chromatography can be performed by standard methods (see, e.g., Ausubel et al., supra). Once isolated, the recombinant protein can, if desired, be purified further by, e.g., high performance liquid chromatography (HPLC; e.g., see Fisher, Laboratory Techniques In Biochemistry and Molecular Biology, Work and Burdon, Eds., Elsevier, 1980).

Polypeptides of the invention, particularly short reptin fragments and longer fragments of the N-terminus and C-terminus of reptin, can also be produced by chemical synthesis (e.g., by the methods described in Solid Phase Peptide Synthesis, 2^{nd} ed., 1984, The Pierce Chemical Co., Rockford, Ill.). These general techniques of polypeptide expression and purification can also be used to produce and isolate useful reptin fragments or analogs, as described herein.

Reptin Protein Fragments

Polypeptide fragments that include various portions of reptin proteins are useful in identifying the domains of reptin that are important for its biological activities. Methods for generating such fragments are well known in the art (see, for example, Ausubel et al., supra), using the nucleotide sequences provided herein. For example, a reptin protein fragment can be generated by PCR amplifying a desired reptin nucleic acid molecule fragment using oligonucleotide primers designed based upon reptin nucleic acid sequences. Preferably, the oligonucleotide primers include unique restriction enzyme sites that facilitate insertion of the amplified fragment into the cloning site of an expression vector (e.g., a mammalian expression vector, see above). This vector can then be introduced into a cell (e.g., a mammalian cell; see above) by artifice, using any of the various techniques that are known in the art, such as those described herein, resulting in the production of a reptin protein fragment in the cell containing the expression vector. Reptin protein fragments (e.g., chimeric fusion proteins) can also be used to raise antibodies specific for various regions of the reptin protein using, for example, the methods described below.

Reptin Protein Antibodies

To prepare polyclonal antibodies, reptin proteins, fragments of reptin proteins, or fusion proteins containing defined portions of reptin proteins can be synthesized in, e.g., bacteria by expression of corresponding DNA sequences contained in a suitable cloning vehicle. Fusion proteins are commonly used as a source of antigen for producing antibodies. Two widely used expression systems for E. coli are lacZ fusions using the pUR series of vectors and trpE fusions using the pATH vectors. The proteins can be purified, coupled to a carrier protein, mixed with Freund's adjuvant to enhance stimulation of the antigenic response in an inoculated animal, and injected into rabbits or other laboratory animals. Alternatively, protein can be isolated from reptin-expressing cultured cells. Following booster injections at bi-weekly intervals, the rabbits or other laboratory animals are then bled and the sera isolated. The sera can be used directly or can be purified prior to use by various methods, including affinity chromatography employing reagents such as Protein A-Sepharose, antigen-Sepharose, and anti-mouse-Ig-Sepharose. The sera can then be used to probe protein extracts from reptin-expressing tissue fractionated by polyacrylamide gel electrophoresis to identify reptin proteins. Alternatively, synthetic peptides can be made that correspond to antigenic portions of the protein and used to inoculate the animals.

To generate peptide or full-length protein for use in making, for example, reptin-specific antibodies, a reptin coding sequence can be expressed as a C-terminal or N-terminal fusion with glutathione S-transferase (GST; Smith et al., Gene 67:31-40, 1988). The fusion protein can be purified on glutathione-Sepharose beads, eluted with glutathione, cleaved with a protease, such as thrombin or Factor-Xa (at the engineered cleavage site), and purified to the degree required to successfully immunize rabbits. Primary immunizations can be carried out with Freund's complete adjuvant and subsequent immunizations performed with Freund's incomplete adjuvant. Antibody titers can be monitored by Western blot and immunoprecipitation analyses using the protease-cleaved reptin fragment of the GST-reptin protein. Immune sera can be affinity purified using CNBr-Sepharose-coupled reptin. Antiserum specificity can be determined using a panel of unrelated GST fusion proteins.

Alternatively, monoclonal reptin antibodies can be produced by using, as an antigen, reptin isolated from reptin-expressing cultured cells or reptin protein isolated from tissues. The cell extracts, or recombinant protein extracts containing reptin, can, for example, be injected with Freund's adjuvant into mice. Several days after being injected, the mouse spleens can be removed, the tissues disaggregated, and the spleen cells suspended in phosphate buffered saline (PBS). The spleen cells serve as a source of lymphocytes, some of which would be producing antibody of the appropriate specificity. These can then be fused with permanently growing myeloma partner cells, and the products of the fusion plated into a number of tissue culture wells in the presence of selective agents, such as hypoxanthine, aminopterine, and thymidine (HAT). The wells can then be screened by ELISA to identify those which contain cells making antibodies capable of binding to reptin, polypeptide fragment, or mutant thereof. These cells can then be re-plated and, after a period of growth, the wells containing these cells can be screened again to identify antibody-producing cells. Several cloning procedures can be carried out until over 90% of the wells contain single clones that are positive for specific antibody production. From this procedure, a stable line of clones that produce the antibody can be established. The monoclonal antibody can then be purified by affinity chromatography using Protein A Sepharose and ion exchange chromatography, as well as variations and combinations of these techniques. Once produced, monoclonal antibodies are also tested for specific reptin recognition by Western blot or immunoprecipitation analysis (see, e.g., Kohler et al., Nature 256:495, 1975; Kohler et al., European Journal of Immunology 6:511, 1976; Kohler et al., European Journal of Immunology 6:292, 1976; Hammerling et al., In Monoclonal Antibodies and T Cell Hybridomas, Elsevier, New York, N.Y., 1981; Ausubel et al., supra).

As an alternate or adjunct immunogen to GST fusion proteins, peptides corresponding to relatively unique hydrophilic regions of reptin can be generated and coupled to keyhole limpet hemocyanin (KLH) through an introduced C-terminal lysine. Antiserum to each of these peptides can be similarly affinity-purified on peptides conjugated to BSA, and specificity tested by ELISA and Western blotting using peptide conjugates, and by Western blotting and immunoprecipitation using reptin, for example, expressed as a GST fusion protein.

Antibodies of the invention can be produced using reptin amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson et al., CABIOS 4:181, 1988. These fragments can be generated by standard techniques, e.g., by PCR, and cloned into the pGEX expression vector. GST fusion proteins can be expressed in E. coli and purified using a glutathione-agarose affinity matrix (Ausubel et al., supra). To generate rabbit polyclonal antibodies, and to minimize the potential for obtaining antisera that is non-specific, or exhibits low-affinity binding to reptin, two or three fusions are generated for each protein, and each fusion is injected into at least two rabbits. Antisera are raised by injections in series, preferably including at least three booster injections.

In addition to intact monoclonal and polyclonal anti-reptin antibodies, the invention features various genetically engineered antibodies, humanized antibodies, and antibody fragments, including F(ab′)2, Fab′, Fab, Fv, and sFv fragments. Truncated versions of monoclonal antibodies, for example, can be produced by recombinant methods in which plasmids are generated that express the desired monoclonal antibody fragment(s) in a suitable host. Antibodies can be humanized by methods known in the art, e.g., monoclonal antibodies with a desired binding specificity can be commercially humanized (Scotgene, Scotland; Oxford Molecular, Palo Alto, Calif.). Fully human antibodies, such as those expressed in transgenic animals, are also included in the invention (Green et al., Nature Genetics 7:13-21, 1994).

Ladner (U.S. Pat. Nos. 4,946,778 and 4,704,692) describes methods for preparing single polypeptide chain antibodies. Ward et al., Nature 341:544-546, 1989, describes the preparation of heavy chain variable domains, which they term “single domain antibodies,” and which have high antigen-binding affinities. McCafferty et al., Nature 348:552-554, 1990, shows that complete antibody V domains can be displayed on the surface of fd bacteriophage, that the phage bind specifically to antigen, and that rare phage (one in a million) can be isolated after affinity chromatography. Boss et al., U.S. Pat. No. 4,816,397, describes various methods for producing immunoglobulins, and immunologically functional fragments thereof, that include at least the variable domains of the heavy and light chains in a single host cell. Cabilly et al., U.S. Pat. No. 4,816,567, describes methods for preparing chimeric antibodies.

Use of Reptin Antibodies

Antibodies to reptin can be used, as noted above, to detect reptin or to inhibit the biological activities of reptin. For example, a nucleic acid molecule encoding an antibody or portion of an antibody can be expressed within a cell to inhibit reptin function. In addition, the antibodies can be coupled to compounds, such as radionuclides and liposomes, for diagnostic or therapeutic uses. Antibodies that inhibit the activity of a reptin polypeptide described herein can also be useful in preventing or slowing the development of a disease caused by inappropriate expression of a wild type or mutant reptin gene.

Detection of Reptin Gene Expression

As noted, the antibodies described above can be used to monitor reptin gene expression. In situ hybridization of RNA can be used to detect the expression of reptin genes. RNA in situ hybridization techniques rely upon the hybridization of a specifically labeled nucleic acid probe to the cellular RNA in individual cells or tissues. Therefore, RNA in situ hybridization is a powerful approach for studying tissue- and temporal-specific gene expression. In this method, oligonucleotides, cloned DNA fragments, or antisense RNA transcripts of cloned DNA fragments corresponding to unique portions of reptin genes are used to detect specific mRNA species, e.g., in the tissues of animals, such as mice, at various developmental stages. Other gene expression detection techniques are known to those of skill in the art and can be employed for detection of reptin gene expression.

Identification of Additional Reptin Genes

Standard techniques, such as the polymerase chain reaction (PCR) and DNA hybridization, can be used to clone reptin gene homologues in other species and reptin-related genes in humans. Reptin-related genes and homologues can be readily identified using low-stringency DNA hybridization or low-stringency PCR with human reptin probes or primers. Degenerate primers encoding human reptin or human reptin-related amino acid sequences can be used to clone additional reptin-related genes and homologues by RT-PCR.

Construction of Transgenic Animals and Knockout Animals

Characterization of reptin genes provides information that allows reptin knockout animal models to be developed by homologous recombination. Preferably, a reptin knockout animal is a mammal, most preferably a mouse. Similarly, animal models of reptin overproduction can be generated by integrating one or more reptin sequences into the genome of an animal, according to standard transgenic techniques. Moreover, the effect of reptin mutations (e.g., dominant gene mutations) can be studied using transgenic mice carrying mutated reptin transgenes or by introducing such mutations into the endogenous reptin gene, using standard homologous recombination techniques.

A replacement-type targeting vector, which can be used to create a knockout model, can be constructed using an isogenic genomic clone, for example, from a mouse strain such as 129/Sv (Stratagene Inc., LaJolla, Calif.). The targeting vector can be introduced into a suitably derived line of embryonic stem (ES) cells by electroporation to generate ES cell lines that carry a profoundly truncated form of a reptin gene. To generate chimeric founder mice, the targeted cell lines are injected into a mouse blastula-stage embryo. Heterozygous offspring can be interbred to homozygosity. Reptin knockout mice provide a tool for studying the role of reptin in embryonic development and in disease. Moreover, such mice provide the means, in vivo, for testing therapeutic compounds for amelioration of diseases or conditions involving reptin-dependent or a reptin-effected pathway.

Use of Reptin as a Marker for Stem Cells of Digestive System Organs or the Heart

As reptin is expressed in cells that give rise to digestive system organs and the heart during the course of development, it can be used as a marker for stem cells of these organs. For example, reptin can be used to identify, sort, or target such stem cells. A pool of candidate cells, for example, can be analyzed for reptin expression, to facilitate the identification of these stem cells, which, based on this identification can be separated from the pool. The isolated stem cells can be used for many purposes that are known to those of skill in this art. For example, the stem cells can be used in the production of new organs, in organ culture, or to fortify damaged or transplanted organs.

Experimental Results

In a genetic screen of chemically mutagenized zebrafish, a recessive mutation (fvo39-K) that leads to impairment of cardiac contractility 72 hours post-fertilization was identified. Affected embryos develop a two-chamber heart with normal appearing cardiac contractility during the first 72 hours of development. However, shortly after this time, decreased ventricular contractility, accompanied by blood congestion in the atrial inflow tract, can be observed. At around 84 hours post-fertilization, the heart stops beating. In addition, at 24 hours post-fertilization, fvo39-k mutant embryos display a body axis deviation (curly down). By 72 hours post-fertilization, eye, jaw, and gut outgrowth fail to occur. These defects become more pronounced as development proceeds.

Histologic analysis of the mutant embryos shows that the most severely affected organs are the eye and the digestive tract. The reduced eye size is due to increased amount of cell death in the retina, which is first detectable at 24 hours post-fertilization using the TUNNEL assay. Liver and exocrine pancreatic cells are also significantly reduced. The mutant intestinal epithelial cells are squamous, unpolarized, pleiomorphic, and reduced in number. Heart morphology appears to be normal at 72 hours post-fertilization. The absence of intestinal terminal differentiation markers, such as intestinal fatty acid binding protein and trypsin, in mutant embryos indicates failure of intestinal and exocrine pancreatic cell terminal differentiation.

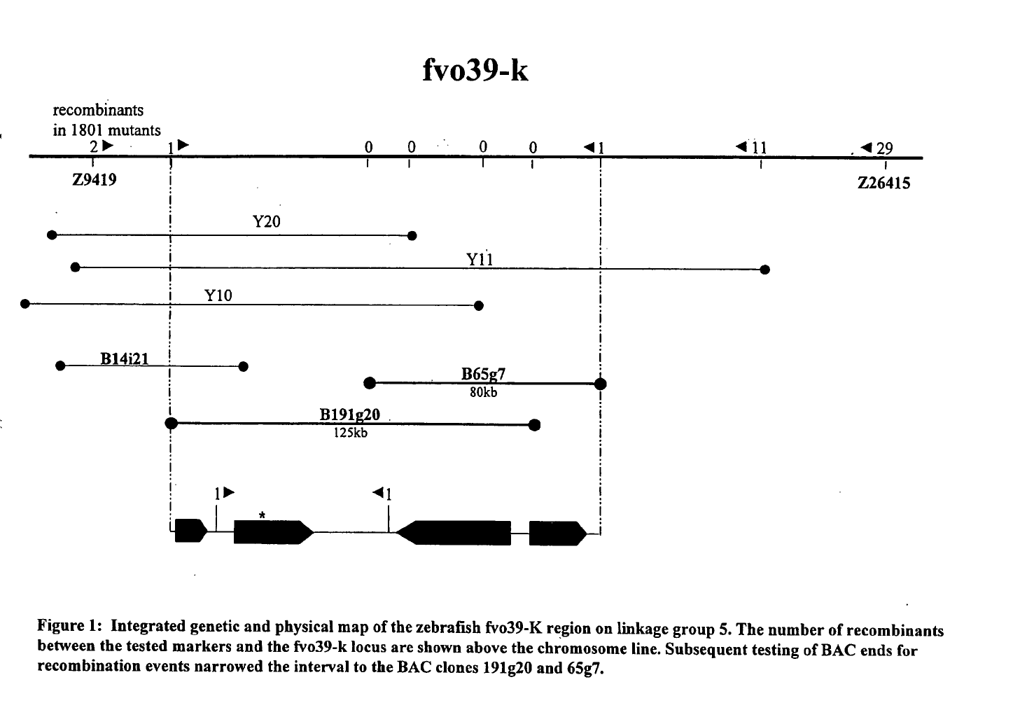

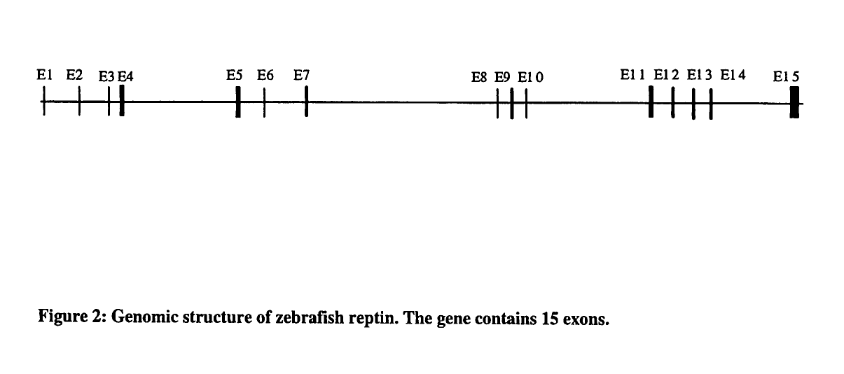

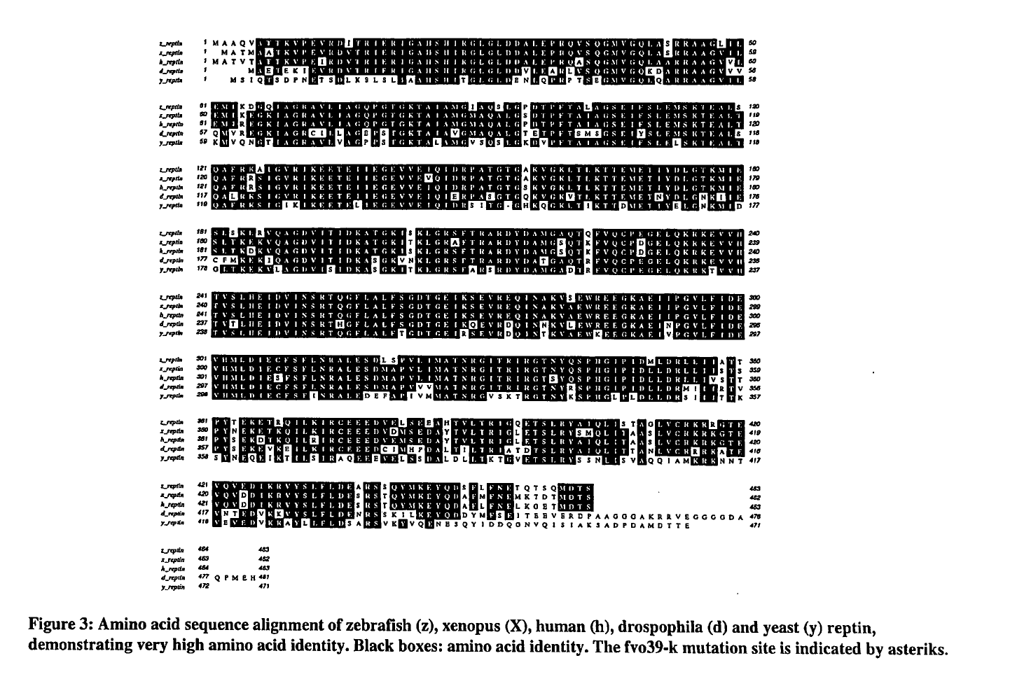

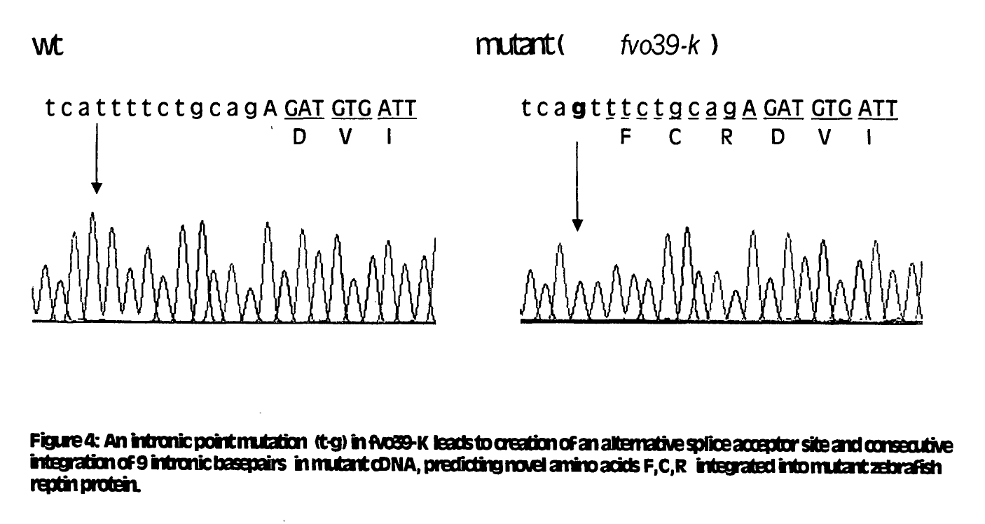

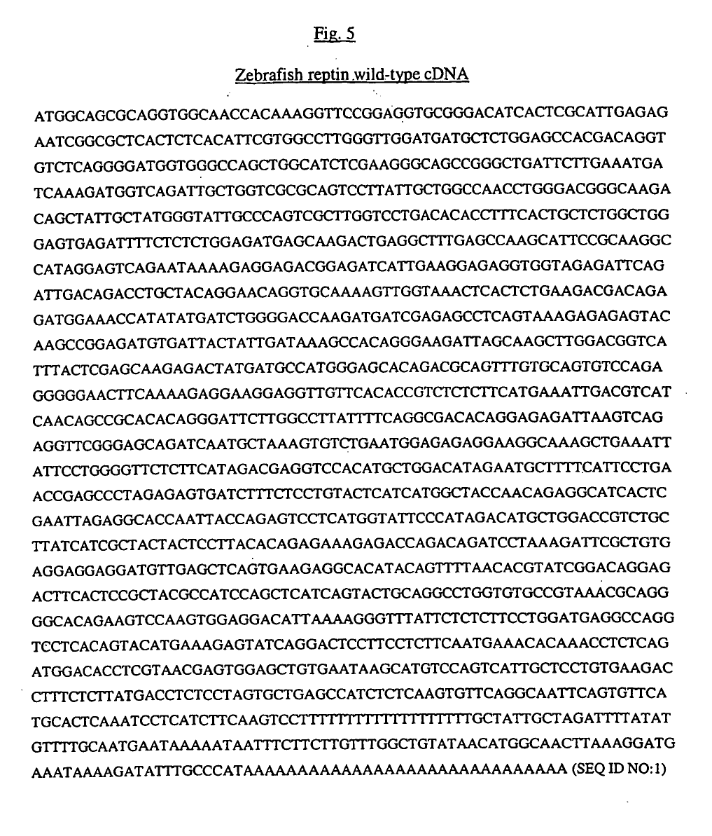

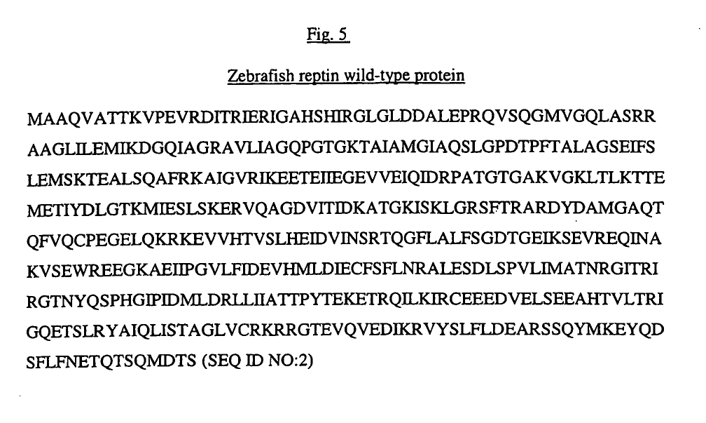

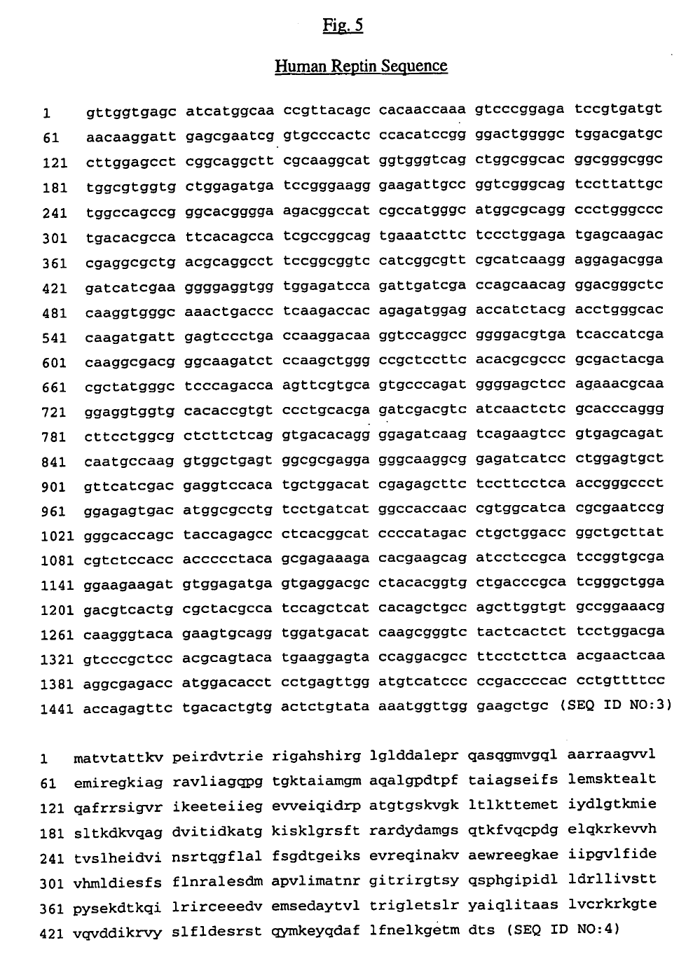

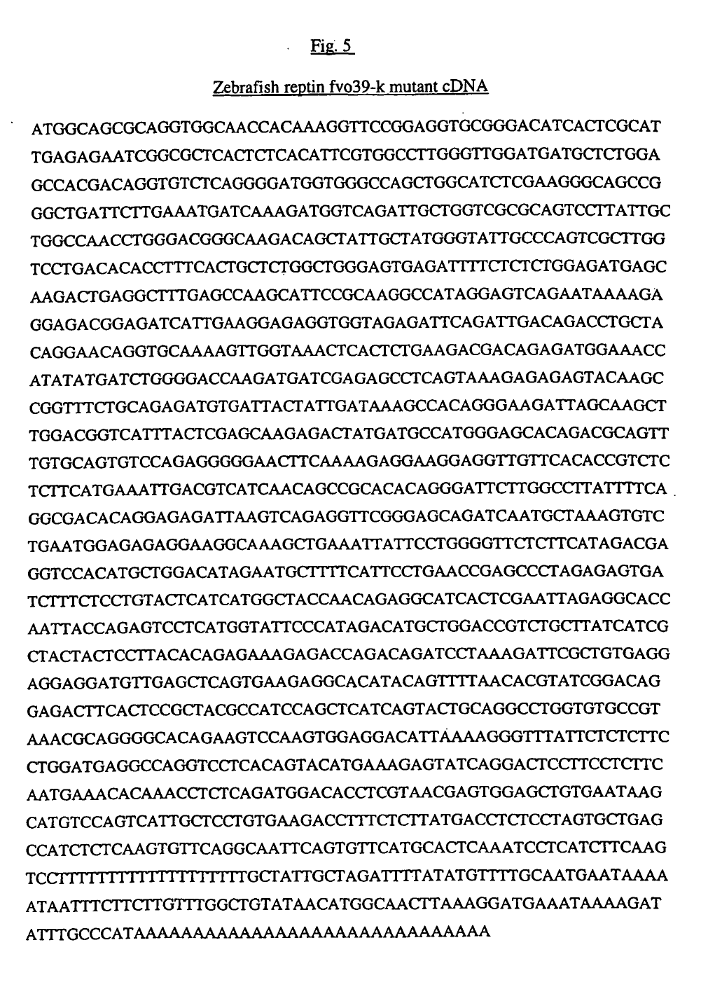

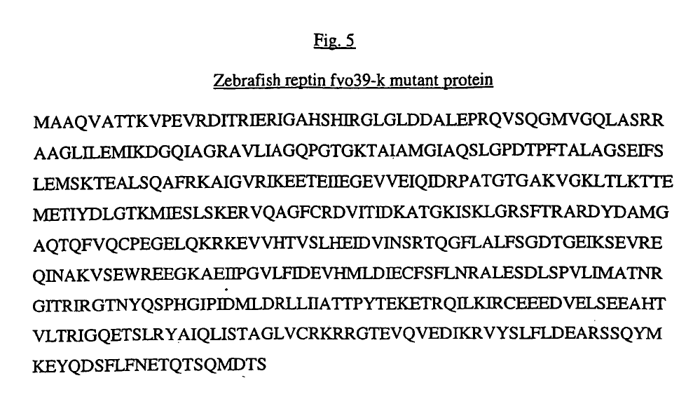

By bulked-segregant analysis, fvo39-k was mapped to zebrafish linkage group 5. Genetic fine-mapping placed the fvo39-k interval between microsatellite markers Z9419 and Z26415 (FIG. 1). The interval was covered with YACs and BACs, and BACs B191g20 and B65g7, containing fvo39-k, were shotgun sequenced. Further genetic fine-mapping restricted the fvo39-k interval to one gene. The intron-exon structure of the gene is illustrated in FIG. 2. BLAST search reveals high homology to Reptin, a RUVB-like DNA helicase (FIG. 3). We thus have designated the gene in which the fvo39-K mutation is present as the zebrafish reptin gene.

Sequencing of zebrafish reptin cDNA from homozygote mutant fvo39-K embryos revealed an insertion of 9 basepairs at the exon7-exon8 boundary. These 9 basepairs encode for new amino acids Phenylalanine (F), Cysteine (C), and Arginine (R). Sequencing of intron7 DNA from mutant embryos revealed a point mutation at position—10(t→g), creating an alternative splice acceptor site. Sequencing of 36 independent cDNA clones demonstrates 100% use of the new splice acceptor site in mutant embryos. Wild-type cDNA could not be detected.

Wild-type and mutant zebrafish reptin RNA was injected into mutant embryos. Embryos were scored for body axis deviation at 48 hours post-fertilization. Injection of wild-type RNA in one-cell embryos can rescue 30% of the mutants (6/20), while injection of mutant RNA rescued 8% (2/25) of mutant embryos.

Morpholino antisense oligonucleotides were designed against the translational start site of zebrafish reptin. Injection of reptin morpholinos phenocopies the fv039-K phenotype. Injection of high doses revealed an even more pronounced effect on body axis and heart morphology and function.

Zebrafish reptin is expressed universally during development, as has been described for Drosophila reptin. However, enhanced expression can be observed at 48 hours post-fertilization in endoderm-derived tissues (branchial arches, liver, pancreas, and gut).

As shown in fvo39-k mutant zebrafish, reptin plays an essential role in brain (retina) and endoderm development, as well as in maintenance of cardiac contractility. Inactivation of the Drosophila orthologue leads to early larval death, indicating an essential and non-redundant function of reptin during early development. Genetic studies in yeast showed that reptin is essential for mitotic growth. Recent work demonstrates that reptin represses transactivation of the β-catenin-TCF complex. β-catenin-mediated transactivation constitutes a mechanism for the control of the canonical Wingless/Wnt pathway. Wnt genes encode evolutionarily conserved secreted proteins, which initiate intracellular transduction cascades critical in numerous developmental processes. Interestingly, the requirement of an intact wnt/β-catenin pathway has been demonstrated in vertebrate body axis formation, brain and craniofacial development, as well as in gut development. The requirement of Wnt signaling for maintaining proper heart contractility in development had not previously been evaluated.

The Wnt signaling pathway has not only been implicated in embryonic patterning, but also in the development of epithelial cancers. For instance, mutations that constitutively activate this pathway are associated with colonic cancer. Therefore, the reptin gene represents a novel target for diagnosing and treating diminished cardiac contractility, digestive organ diseases, and/or intestinal cancers. Since Wnt signaling through β-catenin/Tcf-4 is known to be required for maintenance of an epithelial stem-cell compartment in the murine intestine, modulation of reptin activity can be used to guide endogenous intestinal stem cells into developmental pathways that lead to differentiated organ tissue or, alternatively, for growing organs in culture.

### OTHER EMBODIMENTS

All publications and patent applications mentioned in this specification are herein incorporated by reference to the same extent as if each independent publication or patent application was specifically and individually indicated to be incorporated by reference.

While the invention has been described in connection with specific embodiments thereof, it is to be understood that it is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the appended claims.

1. A method of determining whether a test subject has, or is at risk of developing, a disease or condition related to Reptin, said method comprising analyzing a nucleic acid molecule of a sample from the test subject to determine whether the test subject has a mutation in a gene encoding said Reptin, wherein the presence of a mutation indicates that said test subject has, or is at risk of developing, a disease or condition related to Reptin.

2. The method of claim 1, wherein said test subject is a mammal.

3. The method of claim 1, wherein said test subject is a human.

4. The method of claim 1, wherein said disease or condition is a disease or condition of the heart or the digestive system, or cancer.

5. The method of claim 4, wherein said disease or condition is heart failure.

6. The method of claim 1, wherein said mutation is the fvo39-K mutation.

7. A method for identifying a compound that can be used to treat or to prevent a disease or condition of associated with Reptin, said method comprising contacting an organism comprising a mutation in a gene encoding Reptin and having a phenotype characteristic of a disease or condition associated with Reptin with said compound, and determining the effect of said compound on said phenotype, wherein detection of an improvement in said phenotype indicates the identification of a compound that can be used to treat or to prevent said disease or condition.

8. The method of claim 7, wherein said disease or condition associated with Reptin is a disease or condition of the heart or digestive system, or cancer.

9. The method of claim 7, wherein said organism is a zebrafish.

10. A method of treating or preventing a disease or condition associated with Reptin in a patient, said method comprising administering to said patient a compound identified using the method of claim 7.

11. The method of claim 10, wherein said disease or condition is a disease or condition of the heart or the digestive system, or cancer.

12. The method of claim 10, wherein said patient has a mutation in a gene encoding Reptin.

13. A method of treating or preventing a disease or condition associated with Reptin in a patient, said method comprising administering to said patient a functional Reptin protein or an expression vector comprising a nucleic acid molecule encoding said protein.

14. A method of treating or preventing cancer in a patient, said method comprising administering to said patient a compound or molecule that inhibits the activity or expression of Reptin in said patient.

15. A substantially pure zebrafish Reptin polypeptide.

16. The polypeptide of claim 15, wherein said polypeptide comprises an amino acid sequence that is substantially identical to the amino acid sequence of SEQ ID NO:2.

17. The polypeptide of claim 15, wherein said polypeptide comprises the amino acid sequence of SEQ ID NO:2.

18. A substantially pure polypeptide comprising the sequence of SEQ ID NO:2 and variants thereof comprising sequences that are at least 95% identical to that of SEQ ID NO:2, and which has Reptin activity.

19. An isolated nucleic acid molecule comprising a sequence encoding a zebrafish Reptin polypeptide.

20. The nucleic acid molecule of claim 19, wherein said nucleic acid molecule encodes a polypeptide comprising an amino sequence that is substantially identical to the amino acid sequence of SEQ ID NO:2.

21. The nucleic acid molecule of claim 19, wherein said nucleic acid molecule encodes a polypeptide comprising the amino acid sequence of SEQ ID NO:2.

22. An isolated nucleic acid molecule that specifically hybridizes under high stringency conditions to the complement of the sequence set forth in SEQ ID NO:1, wherein said nucleic acid molecule encodes a protein that has Reptin activity.

23. A vector comprising the nucleic acid molecule of claim 19.

24. A cell comprising the vector of claim 23.

25. A non-human transgenic animal comprising the nucleic acid molecule of claim 19.

26. The non-human transgenic animal of claim 25, wherein said animal is a zebrafish.

27. A non-human animal having a knockout mutation in one or both alleles encoding a Reptin polypeptide.

28. A cell from the non-human knockout animal of claim 27.

29. A non-human transgenic animal comprising a nucleic acid molecule encoding a mutant Reptin polypeptide.

30. The non-human transgenic animal of claim 29, wherein the non-human transgenic animal is a zebrafish.

31. The non-human transgenic animal of claim 30, wherein the non-human transgenic animal comprises the fvo39-K mutation.

32. An antibody that specifically binds to a Reptin polypeptide.

33. A method of modulating the activity of a Reptin polypeptide in a patient, said method comprising administering to the patient an RNA that stimulates or inhibits this activity.
